(19) United States
(12) Patent Application Publication
(10) Pub. No.: US 2025/0134410 A1
(43) Pub. Date: May 1, 2025

(54) MONITORING SYSTEM WITH AN OPTICAL GAS MEASURING DEVICE FOR PILOTS

(71) Applicant: Dräger Safety AG & Co. KGaA, Lübeck (DE)

(72) Inventors: Tilman von BLUMENTHAL, Lübeck (DE); Hans-Ullrich HANSMANN, Lübeck (DE); Henning GERDER, Lübeck (DE); Robert JAHNS, Lübeck (DE); Christoph OSTERLOH, Lübeck (DE)

(21) Appl. No.: 18/925,362

(22) Filed: Oct. 24, 2024

(30) Foreign Application Priority Data
Oct. 25, 2023 (DE) 10 2023 129 369.9

(51) Int. Cl. A61B 5/093

(52) U.S. Cl. CPC A61B 5/093; A61B 2503/22

(57) ABSTRACT

A monitoring system (100) with an associated infrared-optical gas measuring device (72) is used in a breathing gas supply system in aircraft. During a coordinated operation of the monitoring system (100) and the infrared-optical gas measuring device (72), quantities of breathing gas mixture (10) are supplied to the infrared-optical gas measuring device (72). The infrared-optical gas measuring device (72) is configured for metrological detection of a selected target gas with the aid of substitute calibration values (79).

### CROSS REFERENCE TO RELATED APPLICATIONS

This application claims the benefit of priority under 35 U.S.C. § 119 of German Application 10 2023 129 369.9, filed Oct. 25, 2023, the entire contents of which are incorporated herein by reference.

### TECHNICAL FIELD

The present invention relates to a monitoring system with an optical gas measuring device for pilots or passengers of aircraft or aircraft devices. Aircraft or aircraft devices are understood to be airplanes or helicopters in civil or military aviation, such as passenger airplanes in scheduled or charter traffic, as well as ultrafast airplanes close to or above the range of supersonic speed. In particular, flights with jet aircraft at supersonic speeds and/or at altitudes above 15,000 meters above the seabed (sea level) place great demands on the fitness to fly of flight personnel. Fitness to fly with physical and mental fitness, alertness, concentration and vigilance must be ensured at all times at high altitudes, rapid flight maneuvers or flight attitudes, such as turns, dives, inverted flight at high speeds and high accelerations. In addition to the personal and health conditions of the pilot and reliable aircraft equipment, a reliable supply of clean and safe breathing air for the pilot is also essential to ensure safe flight operations.

Breathing air can be supplied by suction from the ambient air-often in the area of the engines or turbines—and/or with a so-called OBOGS system. The supply gas provided in this way is used to supply breathing gas to crews and passengers during a flight with an aircraft. The OBOGS system can be configured as an oxygen enrichment system using an arrangement of molecular sieves based on the principle of pressure swing absorption or as a system with air or pressurized oxygen gas cylinders. As an emergency supply system in the event of a failure of the O₂ main supply, a system for chemical oxygen generation is often also carried on board aircraft. The breathing gas mixture may also be contaminated with other gas components.

### BACKGROUND

U.S. Pat. No. 11,033,762 B2 discloses a device and a method for monitoring inhaled gases from an aircraft pilot's gas supply using an OBOGS system and a face mask.

US 2021 405 008 A1 shows a monitoring system for pilots of an aircraft. The monitoring system makes it possible to monitor the quantities of gas supplied to the pilot with regard to the oxygen or carbon dioxide concentration. For this purpose, a gas mixture from the pilot's face mask is fed to an analyzer by means of a suction measurement.

U.S. Pat. No. 5,026,992 A shows a gas sensor for the optical detection of methane.

A measuring system with a preliminary measurement followed by a specific measurement is described in EP 2 788 751 B1. There, changes in the gas composition in a gas mixture are determined by means of an ultrasonic measurement based on changes in the speed of sound in a gas mixture. Based on this, a measurement with an optical sensor specific to an expected target gas is then activated in order to analyze the gas mixture in relation to the concentration of the target gas.

The present invention relates generally to a gas measuring device for an extended gas analysis of the breathing gas supply of pilots with an on-site/pre-use gas type adjustment or gas type calibration for a monitoring system, as shown, for example, in US 2021 405 008 A1.

Such a monitoring system enables the monitoring of gas quantities of the breathing gas supply of aircraft pilots with regard to the oxygen concentration in the breathing gas during flight operations. For this purpose, a gas mixture from the pilot's face mask and/or from the cockpit is fed to an oxygen sensor in the monitoring system by means of a suction measurement. This oxygen sensor is configured as a paramagnetic oxygen sensor. The paramagnetic oxygen sensor has thermoelectric measuring elements and is thus able to determine the oxygen concentration in the breathing gas by utilizing the paramagnetic effect of the thermal conduction of oxygen on the basis of changes in thermal conductivity under the influence of magnetic field modulation. The utilization of the paramagnetic effect makes the determination of the oxygen concentration in the total mixture of the breathing gas largely independent of the presence of other gases.

From the German patent application DE 10 2023 121 409.8, which has not yet been published (or disclosed), a variant of a monitoring system for monitoring a pilot and/or co-pilot is known, which is configured to determine a condition as to whether there is a presence of foreign gas components in addition to proportions of oxygen, carbon dioxide or nitrogen in a breathing gas mixture. This monitoring system according to DE 10 2023 121 409.8 can determine on the basis of the thermal conductivity information whether a situation exists in which certain quantities of impurities with other gases or gas mixtures are present in the breathing gas mixture. The monitoring system cannot directly determine the nature of the impurities in the breathing gas mixture, but it is possible to identify a particular situation with suspected impurities. Using the paramagnetic measuring device and evaluating thermoelectric voltages, this monitoring system enables an initial analysis of a situation to determine whether additional components such as soot, oil, oil vapors or other gas components are present in the breathing gas mixture in a certain concentration, which are to be regarded as undesirable foreign gases.

For a qualitatively precise determination of the type of impurities in the breathing gas, additional and precise measuring systems, such as a gas chromatograph, must be installed in the aircraft in addition to the monitoring system. However, gas chromatographs cannot be used in aircraft simply because of the confined space and operating requirements. In addition, certain measuring systems with sensors whose functionality is significantly impaired by vibrations, accelerations, extreme and rapid pressure changes caused by large differences in altitude cannot be used sensibly in an aircraft environment. These include, for example, sensors with moving parts, such as chopper wheels, as well as electrochemical sensors or chemical analysis tubes.

### SUMMARY

It is an object of the invention to provide a monitoring system with a further gas measuring device for extended analysis. A further object is to realize a combination of the monitoring system with the further gas measuring device and to specify a coordinated operation of the monitoring system and the gas measuring device.

This problem is solved by a monitoring system for monitoring a gas composition of breathing gases or breathing gas mixtures in a breathing gas supply of aircraft in combination with an infrared-optical gas measuring device that is associated with the monitoring system.

The problem is solved in particular by a monitoring system for extended gas analysis with a paramagnetic measuring device and an optical gas measuring device with features according to the invention.

The present invention supplements the possibilities of the monitoring system shown in US 2021 405 008 A1 with an additional, specially configured gas measuring device in order to realize an extended gas analysis in this way (US20210405008 A1 is incorporated herein by reference). According to the invention, the extended gas analysis by the gas measuring device is carried out using an infrared-optical gas sensor system.

In order to solve the aforementioned problem, a monitoring system for monitoring the gas composition of breathing gases in aircraft or aircraft devices in combination with an infrared-optical gas measuring device is proposed in accordance with the invention. The infrared-optical gas measuring device is characterized by the fact that it can be adjusted in a special way before the start of flight operation, just like the IR gas sensors arranged in the infrared-optical gas measuring device.

The combination of monitoring system and infrared-optical gas measuring device according to the invention enables the infrared-optical gas measuring device to be checked by the control unit of the monitoring system in situations where there may be a presence of foreign gas components in a breathing gas mixture.

The monitoring system has a paramagnetic oxygen sensor, a module for gas transport and a control unit. An infrared-optical gas measuring device with at least one IR gas sensor and an evaluation unit is assigned to the monitoring system.

The control unit is configured to organize, monitor, control or regulate a process of metrological monitoring of the gas composition of air, breathing air or breathing gases in aircraft or aircraft devices.

The control unit can also be configured to coordinate operation of the monitoring system and gas detection device. For this purpose, the control unit of the monitoring system can activate and/or deactivate the infrared-optical gas measuring device.

A gas transport module is provided in the monitoring system, which is configured to draw in defined quantities or partial quantities of breathing gas from a measuring location, in particular at the pilot's breathing mask and/or from the cabin or cockpit, by means of the sample gas line and convey it to the monitoring system. For this purpose, the gas transport module is pneumatically and/or fluidically connected to the measuring point by means of the sample gas line. The quantities of breathing gas mixture extracted by the monitoring system from the breathing mask and/or from the cockpit via a sample gas line are supplied to the infrared-optical gas measuring device and the IR gas sensor as a gas sample by means of a connecting line via the module for gas transport or forwarded to it. The infrared-optical gas measuring device has at least one IR gas sensor. IR gas sensors are particularly suitable for narrow-band detection of certain gases, for example carbon dioxide, propane or methane.

Other gases can also be measured by means of so-called substitute calibration values.

Here is an example: Isopropyl alcohol can be detected with the IR gas sensor if propane is used as the calibration gas in combination with a suitable substitute calibration value for determining the concentration of isopropyl alcohol. This results in pairings of target gases and calibration gases with associated substitute calibration values, which can be documented and/or stored in a database in the form of data records such as tables or data sets, for example. The substitute calibration value includes correlations and/or differences in the optical transmission of IR light from propane and isopropyl alcohol. In this way, a large number of gases or substances in a gas sample can be determined metrologically using the substitute calibration values with just a few types of IR gas sensors. Only in situations in which the selected calibration gas is also present as a component of the gas sample is it not possible to differentiate between the calibration gases used and the target gases.

The present invention takes up this possibility of determining the concentration of other gases deviating from the calibration gas in order to enable the determination of selected other gases in the breathing gas mixture in a monitoring system for aircraft pilots. For this purpose, a combination according to the invention comprising a monitoring system with a paramagnetic oxygen sensor and an infrared-optical gas measuring device with at least one IR gas sensor is suitably configured to detect the proportions of at least one further selected gas or gas mixture in the breathing gas or breathing gas mixture, which is different from oxygen, carbon dioxide and nitrogen, using substitute calibration values. The at least one selected gas or gas mixture can be and is referred to as the target gas in the context of the present invention.

The combination of monitoring system and infrared-optical gas measuring device according to the invention is configured for this purpose as described below.

A module P_{M} for gas transport is configured to supply quantities of breathing gas or breathing gas mixture from a measuring location through a gas inlet on the monitoring system to the paramagnetic oxygen sensor and on to the infrared-optical gas measuring device with the at least one IR gas sensor by means of a sample gas line.

With the paramagnetic oxygen sensor and the control unit, the monitoring system is configured to perform qualitative and quantitative measurement of the concentration of oxygen in the supplied quantities of breathing gas or breathing gas mixture.

The infrared-optical gas measuring device is configured with the at least one IR gas sensor and an evaluation unit for metrological determination of at least one further gas or gas mixture selected as a target gas in the supplied quantities of breathing gas or breathing gas mixture.

A data memory (data storage device) is connected to the evaluation unit. At least one specific substitute calibration value associated with the at least one other gas, gas mixture or group of gases or gas mixtures selected as the target gas is stored as a data record in the data memory.

The evaluation unit is configured to carry out the metrological determination of the presence of the at least one further gas or gas mixture selected as the target gas in the supplied quantities of breathing gas or breathing gas mixture, taking into account (based on) he specific substitute calibration value assigned to the at least one further gas, gas mixture or group of gases or gas mixtures selected as the target gas.

The evaluation unit is also configured to determine a target gas identifier, which indicates the presence of the target gas in the breathing gas mixture, as an output signal. The target gas identifier can comprise a concentration, a volume concentration, a volume fraction, a volume ratio, a substance concentration (molarity), a weight fraction or a partial pressure of the target gas in the breathing gas mixture.

Embodiments can show how an initiation and/or control of the supply of the gas sample to the optical gas measuring device can be configured. Such embodiments relate, for example, to the switching or changeover of valves and/or changeover valves as well as activations or deactivations of the module for gas transport as well as of further gas conveying means arranged in the gas measuring device, for example pumps, in order to convey the quantities of breathing gas or breathing gas mixture as a gas sample from the monitoring system to the infrared-optical gas measuring device.

Embodiments can show how the infrared-optical gas measuring device can be connected to the monitoring system. For example, the infrared-optical gas measuring device can be integrated as a module in the monitoring system as well as arranged as a further, additional module at the gas outlet of the monitoring system.

The supply of the gas sample to the infrared-optical gas measuring device can be controlled by the control unit, for example by means of an actively switched valve. In alternative embodiments, gas samples from the face mask and/or the cockpit can be supplied in parallel to both the monitoring system with the paramagnetic measuring system and the infrared-optical gas measuring device at the same time.

The use of surrogate calibration values in the infrared optical gas detector simplifies the user's search for a specific gas in preparation for flight operations by allowing the user to perform a calibration with selection and/or entry of a surrogate calibration value or a selection from a plurality of surrogate calibration values before operating the aircraft. First, a target gas is selected, which may be present as a component in the breathing gas mixture during flight operations. Then the calibration gas is selected, for example propane, nonane, hexane, butane, usually methane, for which a suitable substitute calibration value is available in the pairing of calibration gas and target gas. The gas measuring system is then calibrated with the calibration gas-if this has not already been done in preparation for flight operations when the monitoring system and infrared-optical gas measuring device are being upgraded. Finally, the substitute calibration value for the selected pairing of target gas and calibration gas is provided to the monitoring system and/or the infrared-optical gas measuring device. The infrared-optical gas measuring device is thus suitably equipped to qualitatively and quantitatively measure the target gas, determine the target gas identification and provide it as an output signal if the target gas occurs in the breathing gas mixture during subsequent flight operations. Knowing the auxiliary and operating materials used in the Aircraft or aircraft device in different constellations, the IR gas sensors can be advantageously set, i.e. adjusted, to known auxiliary or operating materials with the aid of the substitute calibration values. This means that the user can configure the IR gas sensors to an expected target gas for the upcoming use-even directly on site-without having to know or disclose information on auxiliary or operating materials for the design, development and provision of the monitoring system. Examples of auxiliary or operating materials include fuel, coolants, lubricants, lubricants, coatings or paints. The operator of the aircraft or aircraft device can therefore configure the IR gas sensors for substances before use without having to provide a manufacturer of the monitoring system or a manufacturer of the gas measuring device with information about the type of substance in question. This type of on-site configuration enables the operator to comply with any existing confidentiality requirements vis-à-vis third parties with regard to specific expected target gases if, for example, the auxiliary materials or fuels for aircraft are a possible source of the target gases and these auxiliary materials or fuels are based on special and possibly not freely available formulations.

The at least one IR gas sensor is preferably closely coupled to the monitoring system by means of a data connection, for example in the form of a bus system, and preferably also connected to its power supply. Since the monitoring system is preferably configured to be mobile and independent of the aircraft and is arranged on the clothing of the pilot, e.g. in a vest or jacket pocket, it is essential to use the amount of energy carried in the monitoring system as sparingly as possible. It makes sense that only those components of the monitoring system and/or the gas detection device that are absolutely necessary for the respective task are continuously in operation and/or electrically supplied. Optical gas sensors cannot necessarily be implemented as low-power sensors due to the measuring principles with a radiation source and sensor heating to prevent moisture condensation.

To ensure careful handling of the stored electrical energy, which is only available for a limited time during the period of use, it is therefore advantageous to carry out neither the supply of a gas sample to the other sensors, nor the measurement operation, data acquisition or data storage continuously, but only when an exceptional situation occurs that is detected by the monitoring system or based on a special situation, such as an activity of the aircraft crew.

If an unusual or special situation arises, the control unit in the monitoring system can then activate a valve arrangement and/or pump arrangement in such a way that gas quantities are conveyed to the additional sensor system with the infrared optical gas sensor.

At the same time, the additional sensor system is then activated and the gas sample is then analyzed in relation to the unusual or special situation. When the sensor system with the infrared-optical gas sensor is activated, the control unit in the monitoring system can also switch on the power supply for the other sensor system and/or the infrared-optical gas sensor as required.

In a preferred embodiment, the control unit is configured to carry out a determination as a function of (based on) the target gas detection provided by the gas measuring device as to whether there is a current situation that poses a health risk to an aircraft pilot during operation of the breathing gas supply of the aircraft. If a situation that poses a health risk to the pilot is currently present, the control unit is configured to activate a vibration alarm detector arranged in or on the monitoring system or a vibration alarm detector assigned to the monitoring system. The vibration detector can be integrated in a housing of a monitoring system configured as a mobile unit or a mobile gas measuring device. In an alternative embodiment, the vibration detector can also be configured as a component in a wristwatch or another mobile device arranged on the wrist of a pilot or co-pilot. In addition to the vibration detector, the wristwatch or the other mobile device arranged on the wrist of a pilot or co-pilot can also have elements for a visual or acoustic alarm of the health-threatening situation and thus be configured to provide an acoustic and/or visual and/or tactile alarm.

In further preferred embodiments, components of the monitoring system and the infrared-optical gas measuring device can be connected to each other by means of an interface arrangement comprising one or more interfaces and line connections. For example, the control unit can also be configured to coordinate the operation of the infrared-optical gas measuring device and the monitoring system by means of the one or more interfaces and the line connections.

If, for example, the control unit in the monitoring system, in conjunction with the paramagnetic oxygen sensor, has detected a condition through a special evaluation of thermal conductivity situations—as described, for example, in DE 10 2023 121 409.8 (corresponding U.S. application Ser. No. 18/798,948 is incorporated herein by reference)—a condition has been detected that a gas or gas mixture other than oxygen, nitrogen, moisture or water vapor and carbon dioxide may be present in the breathing gas mixture, the control unit can on the one hand activate the IR-optical gas measuring device, i.e. put it into measuring mode, and/or activate switching or changeover device in the monitoring system and/or infrared-optical gas measuring device in such a way that a gas sample of breathing gas can be supplied to the IR gas sensor.

In particularly preferred embodiments, the control unit can be configured to perform at least one action of the following actions when performing the coordination of the monitoring system and the infrared-optical gas measuring device:
Activation of a switching device (switching means) or a changeover device (changeover means) configured to start the supply of quantities of breathing gas to the infrared-optical gas measuring device;
Deactivation of switching device or changeover device to terminate the supply of quantities of breathing gas to the infrared-optical gas measuring device;
Activation of components of the infrared-optical gas measuring system such as evaluation unit, IR gas sensor, components for electrical power supply electrical power supply;
Deactivation of components of the infrared-optical gas measuring device such as evaluation unit, IR gas sensor, components for electrical power supply;
Connection of the electrical power supply of the monitoring system and/or gas detection device or individual components of the monitoring system and/or infrared-optical gas measuring device; and
Switching off the electrical power supply to the monitoring system and/or gas detection system or individual components of the monitoring system and/or infrared-optical gas measuring device.

If the additional sensor system is configured as an independent module with its own power supply, the control unit in the monitoring system can activate the infrared-optical gas measuring device from a standby or idle mode with low energy requirements into a measuring mode, in which the IR gas sensor is then also supplied with electrical energy. Through such measures of energy management and energy control by the control unit in the monitoring system, the energy storage units of the monitoring system and/or the other sensors required for flight operation can be kept as small as possible in terms of weight, volume and size. This is particularly advantageous for mobile equipment worn on the body or close to the body, for example in terms of wearing comfort and space requirements on clothing and with regard to minimizing restrictions on the pilot's mobility at his workplace. In the event of an unplanned exit from the cockpit using a parachute, it is also advantageous to largely avoid any unnecessary weight on the pilot's clothing as well as restrictions on the pilot's mobility on the parachute.

In a further preferred embodiment, an input unit can be provided on the monitoring system and/or on the infrared-optical gas measuring device, which enables a selected gas or gas mixture to be entered or selected as the target gas and/or a calibration gas and/or the specific substitute calibration value.

In a further preferred embodiment, an interface (interface arrangement) can be provided on the monitoring system and/or on the infrared-optical gas measuring device, which enables the provision of special information on a target gas, on a calibration gas and/or on a substitute calibration value indexed to the infrared-optical gas measuring device.

By providing the special information via an interface (interface arrangement) or selecting a target gas at the input unit, the infrared-optical gas measuring device is prepared for a measuring operation in which qualitative and/or quantitative metrological detection of the target gas is possible.

In a further preferred embodiment, an output unit can be arranged in or on the monitoring system and/or in or on the infrared-optical gas measuring device, which is configured to provide and/or visually output the selected target gas and/or to output status information relating to the infrared-optical gas measuring device. This allows measured values or gas concentration values of the target gas as well as status information during operation of the infrared-optical gas measuring device, such as standby or idle mode, active measuring mode, current energy consumption, alarm statuses as well as a charge status of the energy stores to be output to the user as visual messages, for example alphanumerically, in text form or graphical form (GUI).

In further preferred embodiments, substitute calibration values for various gases or gas mixtures can be stored in the data memory as target gases based on the calibration gases propane, nonane, hexane, methane, butane, pentane in data records (such as data sets).

The data records can be stored in tables, data sets or database systems in a sortable manner based on the target gases or calibration gases. The data memory can also be configured as an external data memory, for example as a USB stick, so that during the configuration of the infrared-optical gas measuring device, information is imported from the external data memory into the evaluation unit of the infrared-optical gas measuring device in a manner adapted to the subsequent use and the data memory itself is no longer required in subsequent operation. This type of on-site configuration enables a robust design of the operation of the infrared-optical gas measuring device with regard to possible secrecy requirements with regard to the potentially expected target gases.

The invention is explained in more detail in the following description with partial reference to the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a monitoring system with features forming a part of a system with the monitoring system and an infrared-optical gas measuring device according to the invention; and

FIG. 2 is a schematic view showing features of the monitoring system with the infrared-optical gas measuring device.

### DESCRIPTION OF PREFERRED EMBODIMENTS

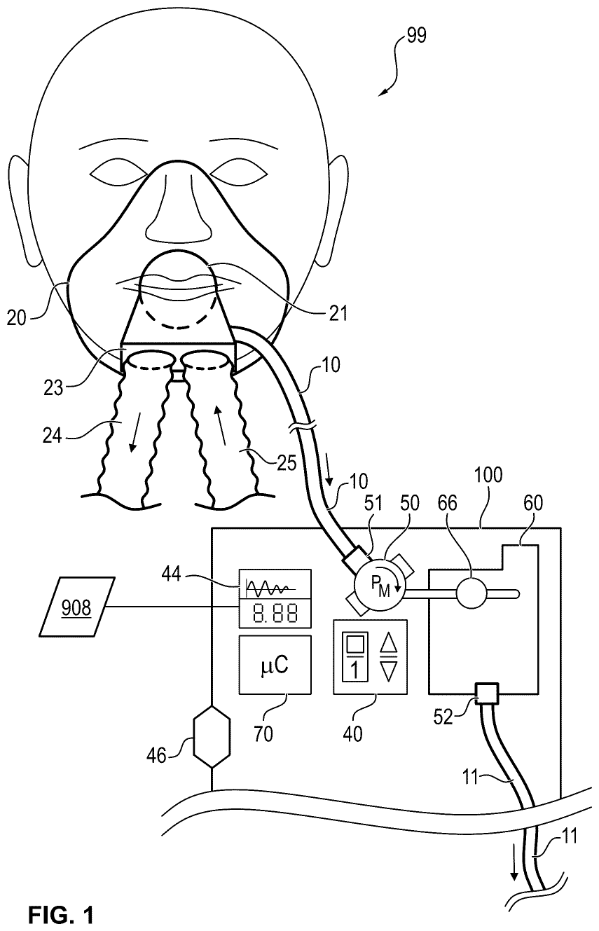

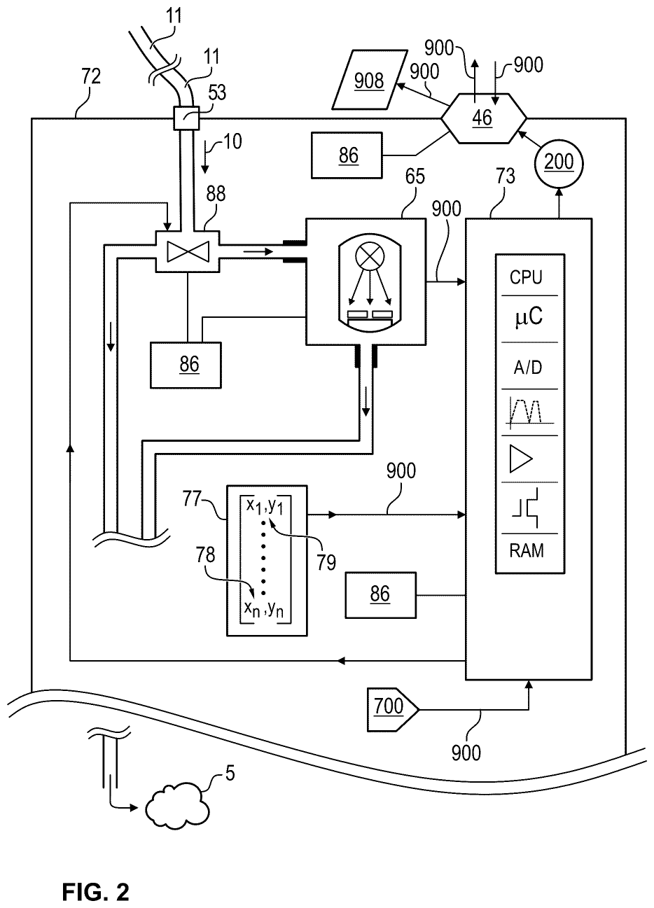

Referring to the drawings, identical elements in FIGS. 1 and 2 are designated with identical reference numerals in FIGS. 1 and 2. In the description of FIGS. 1 and 2, reference is made to each other to explain the overall functionality of the breathing mask, monitoring system and infrared-optical gas measuring device.

FIG. 1 shows a schematic representation of a monitoring system 100 with some features according to US20210405008 A1 along with input elements 40, display elements 44, control unit 70, interface 46, output signal 908, which enables control and/or configuration of the operation of monitoring system and gas measuring device, as well as connection features including gas outlet 52 and a connecting line 11 to form a part of a monitoring system and gas measuring device system according to the invention.

The monitoring system 100 is connected to a person 99 via a sample gas line 10 by means of a breathing mask 20. The person 99 is an aircraft pilot. The breathing mask 20 has a gas connection 21, a connection element 23 and hose lines 24, 25. The hose lines 24, 25 are used for the removal and supply of breathing gases to the person 99. The monitoring system 100 has operating and input elements 40, display elements 44, at least one module for gas delivery (gas transport module) 50, a sensor system 60 with at least one sensor 66. The at least one sensor 66 is configured as a paramagnetic gas sensor. The module for gas delivery 50 is preferably configured as a pump P_{M}. In addition, the monitoring system 100 has a control unit 70. The operating and input elements 40, the display elements 44, the sensor system 60 and the gas delivery module 50 are connected to the control unit 70 via signal and data lines or control lines, which are not shown in FIGS. 1 to 2. These control lines or signal and data lines can, for example, be configured as a bus system or network. The control unit 70 is configured and intended to control and/or actuate the gas delivery module 50 in such a way that breathing gases or breathing gas mixtures are delivered from the breathing mask 20 through the sample gas line 10 and a gas inlet 51 to the sensor system 60. Thus, a quantity or partial quantity of breathing gas mixture 10 is then available to the at least one sensor 66 in order to detect and/or analyze it metrologically and provide it to the control unit 70 as measured values. The control unit 70 enables the measured values to be evaluated, processed and displayed on display elements 44. A quantity of breathing gas 10 is forwarded to a gas inlet 53 of an infrared-optical gas measuring device 72 via a schematically shown gas outlet 52 and a connecting line 11. The operating and input elements 40 make it possible to configure the monitoring system 100 and/or the gas measuring device 72 of the monitoring system 100 and the gas measuring device 72. In addition, an interface arrangement comprising the interface 46 is provided on the gas measuring device, which enables control and/or configuration of the operation 900 of monitoring system 100 and gas measuring device 72.

FIG. 2 shows the monitoring system 100 according to FIG. 1 in combination with an infrared-optical gas measuring device 72. The infrared-optical gas measuring device 72 has an IR gas sensor 65. Quantities of breathing gas mixture 10 can flow as a gas sample via the connecting line 11 from the monitoring system 100 via a gas inlet 53 to the infrared-optical gas measuring device 72 and with the aid of switching device and/or changeover device 88, which can be in the form of valves or ⅔-way valves, for example, to the IR gas sensor 65. According to this FIG. 2, an evaluation unit 73 is configured as a submodule of the control unit 70 of the monitoring system 100 by means of the interface arrangement comprising at least one interface 46 to carry out coordination 900 and cooperation between the monitoring system 100 and the gas measuring device 72 as well as an exchange of data and/or instructions with the evaluation unit 73 via the interface 46. A data memory 77 is assigned to the evaluation unit 73. Substitute calibration values 79 for a large number of constellations of calibration gases and target gases can be stored as data records such as data sets 78 in the data memory 77. The infrared-optical gas measuring device 72 is configured with the at least one IR gas sensor 65 and the evaluation unit 73 for a metrological determination of at least one further gas or gas mixture selected as a target gas in the supplied quantities of breathing gas or breathing gas mixture 10. The evaluation unit 73 is furthermore configured to carry out the metrological determination of a presence of the at least one further gas or gas mixture selected as a target gas in the supplied quantities of breathing gas or breathing gas mixture 10, based on (taking into account) the specific substitute calibration value 79 assigned to the at least one further gas, gas mixture or group of gases or gas mixtures selected as a target gas and stored as data set 78 in the data memory 77. Finally, the evaluation unit 72 is configured to determine a target gas identifier 200, which indicates the presence of the target gas in the breathing gas mixture 10, and to provide the target gas identifier 200 as an output signal 908 at the output unit 44 and/or the interface 46.

In addition, it is possible for the control unit 70 or the evaluation unit 73 to include special conditions 700 in the control and coordination of the module 50 for gas delivery, the switching or changeover device 88 when supplying quantities of breathing gas mixture 10 to the gas measuring device 72 to the IR gas sensor 65. Such situations include, for example, conditions 700 in which it may be suspected that a gas or gas mixture other than oxygen, nitrogen, moisture or water vapor and carbon dioxide may be present in the breathing gas mixture 10. Such special situations 700 can serve the control unit 70 or the evaluation unit 73 as a trigger event in order to activate the switching device or switching device 88 as well as components of a power supply 86 of the gas measuring device 72 in such a way as to enable a supply of quantities of breathing gas mixture 10 to the IR gas sensor 65 and to initiate analysis and metrological determination of the target gas. By means of the switching device or changeover device 88, for example, quantities of breathing gas mixture 10 can be distributed and/or split up as a gas sample between the IR gas sensor 65 and a feed-through or discharge by means of a connecting line 12 into an environment 5, for example a cockpit of an aircraft.

While specific embodiments of the invention have been shown and described in detail to illustrate the application of the principles of the invention, it will be understood that the invention may be embodied otherwise without departing from such principles.

### REFERENCE NUMBER LIST

5 Surroundings
10 Sample line (sample gas line)
11, 12 Connecting cables
20 Breathing mask
21 Gas connection to breathing mask
24, 25 Hose lines
40 Control elements
44 Display elements
46 Interface arrangement with one or more interfaces
50 Module for gas delivery (gas pumping module), pump P_{M}
51, 53 Gas inlet
52 Gas outlet
60 Sensors
65 IR optical gas sensor
66 Gas sensor, paramagnetic oxygen sensor
70 Control unit, control module
72 Gas measuring device
73 Evaluation unit
77 Data memory
78 Data set, data sets
79 Substitute calibration value
86 Components of the electrical power supply
88 Switching device, valves
99 Person, pilot, co-pilot, aircraft pilot
100 Monitoring system
200 Target gas identification
700 Special condition, situation
900 Operation, control and coordination of operations
908 Output signal

What is claimed is:

1. A system comprising:
a monitoring system for monitoring a gas composition of breathing gases or breathing gas mixtures in a breathing gas supply of aircraft, the monitoring system comprising: a paramagnetic oxygen sensor; a gas transport module for gas transport; and a control unit;
an infrared-optical gas measuring device associated with the monitoring system, the infrared-optical gas measuring device comprising: an IR gas sensor; an evaluation unit; and a data memory connected to the evaluation unit,
wherein the control unit is configured to organize, monitor, control or regulate a process of metrological monitoring of the gas composition of air, breathing air, breathing gases or breathing gas mixture in aircraft or flying devices and to coordinate operation of the monitoring system and the infrared-optical gas measuring device,
wherein the gas transport module is configured for gas transport by means of a sample gas line for supplying quantities of breathing gas or breathing gas mixture from a measuring location through a gas inlet of the monitoring system to the paramagnetic oxygen sensor and to the infrared-optical gas measuring device,
wherein the monitoring system with the paramagnetic oxygen sensor and the control unit is configured to perform a qualitative and a quantitative metrological detection of a concentration of oxygen in the supplied quantities of breathing gas or breathing gas mixture,
wherein the infrared-optical gas measuring device and the evaluation unit are configured for a metrological determination of at least one further gas or gas mixture selected as a target gas in the supplied quantities of breathing gas or breathing gas mixture,
wherein at least one specific substitute calibration value for at least one target gas associated with the at least one further gas, gas mixture or group of gases or gas mixtures selected as target gas is stored in the data memory as a data record,
wherein the evaluation unit is configured to carry out the metrological determination of the presence of the selected at least one further gas or gas mixture selected as target gas in the supplied quantities of breathing gas or breathing gas mixture, based on the specific substitute calibration value assigned to the at least one further gas, gas mixture or group of gases or gas mixtures selected as target gas, and
wherein the evaluation unit is configured to determine a target gas identifier, which indicates the presence of the target gas in the breathing gas mixture, and to provide the target gas identifier as an output signal.

2. A system according to claim 1,
wherein the control unit is configured to determine, depending on the target gas identifier provided by the gas measuring device, whether a current health-endangering situation for an aircraft pilot exists during operation of the breathing gas supply of the aircraft, and
wherein, upon a determination that the health-endangering situation for an aircraft pilot currently exists, the control unit is configured to activate a vibration alarm detector arranged in or on the monitoring system or a vibration alarm associated with the monitoring system.

3. A system according to claim 1, further comprising an interface arrangement comprising one or more interfaces and line connections configured to connect components of the monitoring system and the infrared-optical gas measuring device to one another, wherein the control unit is configured to coordinate operation of the infrared-optical gas measuring device and the monitoring system by means of the interface arrangement.

4. A system according to claim 1, further comprising a switching device or changeover device configured to supply quantities of breathing gas to the infrared-optical gas measuring device, wherein the control unit is configured, for coordinating the monitoring system and the infrared-optical gas measuring device, to execute an activation of the switching device or the changeover device for starting the supply of quantities of breathing gas to the infrared-optical gas measuring device and to execute a deactivation of switching device or the changeover device for terminating the supply of quantities of breathing gas to the infrared-optical gas measuring device.

5. A system according to claim 1, wherein the control unit is configured, for coordinating the monitoring system and the infrared-optical gas measuring device, to execute an activation of components of the infrared-optical gas measuring device, wherein the components comprise one or more of the evaluation unit, the IR gas sensor and an electrical power supply, and to execute a deactivation of the components of the infrared-optical gas measuring device.

6. A system according to claim 1,
wherein the control unit is configured, for coordinating the monitoring system and the infrared-optical gas measuring device, to execute a connection of an electrical power supply of the monitoring system and/or gas measuring device or individual components of the monitoring system and/or infrared-optical gas measuring device; or
wherein the control unit is configured, for coordinating the monitoring system and the infrared-optical gas measuring device, to switch off the electrical power supply of the monitoring system and/or gas detection device or individual components of the monitoring system and/or the infrared-optical gas measuring device.

7. A system according to claim 1, wherein the monitoring system and/or on the infrared-optical gas measuring device further comprises an input unit, the input unit being configured to enables an input or a selection of a selected gas or gas mixture as target gas and/or a calibration gas and/or the specific substitute calibration value.

8. A system according to claim 1, wherein the monitoring system and/or on the infrared-optical gas measuring device further comprises an interface, the interface being configured to indicate and enable the provision of information on a target gas, on a calibration gas and/or on a substitute calibration value to the infrared-optical gas measuring device.

9. A system according to claim 1, wherein the monitoring system and/or on the infrared-optical gas measuring device further comprises an output unit, the output unit being assigned to the monitoring system and/or the infrared-optical gas measuring device and being configured to provide and/or visually output in relation to the selected target gas and/or to output status information in relation to the infrared-optical gas measuring device.

10. A system according to claim 1, wherein respective substitute calibration values are based on the gases propane, nonane, hexane, methane, butane, pentane and are stored in data sets in the data memory.

11. A system according to claim 1, wherein the evaluation unit is configured as a submodule of the control unit of the monitoring system.

12. A system according to claim 1, wherein the infrared-optical gas measuring device is integrated in the monitoring system as a module of the monitoring system or is arranged as a further, additional module, at the gas outlet of the monitoring system.
